(19) United States
(12) Patent Application Publication
(10) Pub. No.: US 2019/0247063 A1
(43) Pub. Date: Aug. 15, 2019

(54) ORTHOPAEDIC SURGICAL INSTRUMENT SYSTEM AND METHOD FOR PREPARING A PATIENT'S CALCAR

(71) Applicant: DEPUY SYNTHES PRODUCTS, INC., RAYNHAM, MA (US)

(72) Inventors: Daniel N. Huff, Warsaw, IN (US); Jeffrey A. McAnelly, Warsaw, IN (US); Ian G. Delaney, Warsaw, IN (US)

(21) Appl. No.: 15/897,306

(22) Filed: Feb. 15, 2018

(51) Int. Cl. A61B 17/17; A61B 17/16; A61F 2/36

(52) U.S. Cl. CPC A61B 17/175; A61F 2/3601; A61B 17/1668

(57) ABSTRACT

An orthopaedic surgical instrument system including a reamer assembly is disclosed. The reamer assembly includes a reamer head having a plurality of cutting teeth and a mounting bracket configured to be coupled to a femoral broach, which is sized to be positioned in a proximal opening of a femoral canal of a patient's femur. When the mounting bracket is coupled to the femoral broach, the reamer head is offset medially from the lateral edge of the femoral broach to resect a medial calcar of the patient femur.

### TECHNICAL FIELD

The present disclosure relates generally to orthopaedic prostheses, and particularly to orthopaedic prostheses for use in hip replacement surgery.

### BACKGROUND

Joint arthroplasty is a well-known surgical procedure by which a diseased and/or damaged natural joint is replaced by a prosthetic joint. The prosthetic joint may include a prosthesis that is implanted into one or more of the patient's bones. Many hip prostheses include a femoral prosthesis that is implanted into a patient's femur. A femoral prosthesis typically includes a femoral stem component that is received in the patient's femur and a spherically-shaped head component that bears against the patient's acetabulum or a prosthetic replacement acetabular cup.

Femoral stem components typically include an elongated body that is sized and shaped to be inserted into a surgically-prepared femoral canal. Some femoral stem components include a medial collar positioned at the proximal end of the elongated body. The medial collar engages a surgically-prepared calcar of the patient's femur to modify how loads placed on the femoral prosthesis are transmitted to the patient's femur.

To prepare a patient's femur to receive a stem component, a surgeon or other member of the surgical team may use a variety of orthopaedic surgical instruments, including cutting saws, osteotomes, and femoral broaches. For collared stem components, a calcar reamer may also be used to prepare the patient's femur for the medial collar.

### SUMMARY

According to one aspect of the disclosure, an orthopaedic surgical instrument system is disclosed. The system includes a reamer head having a plurality of cutting teeth and a mounting bracket that is coupled to the reamer head and is configured to be coupled to a femoral broach or other surgical instrument. When the mounting bracket is coupled to the femoral broach, the reamer head is offset medially from the lateral edge of the femoral broach to resect a medial calcar of the patient femur.

According to another aspect, the orthopaedic surgical instrument system comprises a first orthopaedic surgical instrument including an elongated body sized to be positioned in a femoral canal of a patient's femur and a plurality of cutting teeth defined in the elongated body. The elongated body includes a medial edge and a lateral edge positioned opposite the medial edge, and, in some embodiments, the cutting teeth are defined along the length of the medial and lateral edges. The orthopaedic surgical instrument system also comprises a second orthopaedic surgical instrument including a reamer head configured to resect a portion of a proximal end of the patient's femur and a mounting bracket configured to secure the reamer head to a proximal end of the first orthopaedic surgical instrument. When the mounting bracket is coupled to the proximal end of the first orthopaedic surgical instrument, an outer circumference of the reamer head is offset medially from the lateral edge of the elongated body such that the reamer head is positioned to resect a medial calcar of the patient's femur.

In some embodiments, the reamer head may be positioned above only the medial edge of the elongated body when the mounting bracket is coupled to the proximal end of the first orthopaedic surgical instrument.

In some embodiments, the reamer head may be coupled to the mounting bracket such that the reamer head is permitted to move in an inferior-superior direction relative to the elongated body of the first orthopaedic surgical instrument when the mounting bracket is coupled to the proximal end of the first orthopaedic surgical instrument.

In some embodiments, the elongated body of the first orthopaedic surgical instrument may include a proximal planar surface that has a medial-lateral width, and the outer circumference of the reamer head may have a diameter less than the medial-lateral width of the proximal planar surface.

Additionally, in some embodiments, the first orthopaedic surgical instrument may include a post extending outwardly from the proximal planar surface, and the mounting bracket may be configured to be coupled to the post. In some embodiments, the first orthopaedic surgical instrument may include a slot defined in the proximal planar surface, and the mounting bracket of the second orthopaedic surgical instrument may include a tab sized to be received in the slot to prevent rotation of the mounting bracket relative to the mounting bracket relative to the first orthopaedic surgical instrument.

In some embodiments, the mounting bracket of the second orthopaedic surgical instrument may include a cylindrical passageway that is sized to receive the post. Additionally, in some embodiments, the cylindrical passageway may extend along a longitudinal axis, and the reamer head may be configured to rotate about a rotational axis extending parallel to the longitudinal axis. In some embodiments, the second orthopaedic surgical instrument may include an elongated shaft extending along the rotational axis from the reamer head to a shank shaped to be coupled to a rotary surgical instrument.

In some embodiments, the second orthopaedic surgical instrument may include a housing connected to the mounting bracket, and the elongated shaft of the second orthopaedic surgical instrument may extend through the housing to the shank positioned outside of the housing.

In some embodiments, the first orthopaedic surgical instrument may be a femoral broach. In some embodiments, the medial edge of the elongated body of the first orthopaedic surgical instrument may include a concave section, and the lateral edge of the elongated body may include a convex section positioned opposite the concave section.

In some embodiments, the elongated body may extend to a distal tip.

According to another aspect, an orthopaedic surgical instrument system comprises a housing extending along a first longitudinal axis from a superior end to an inferior end and a mounting bracket extending from the inferior end of the housing. The mounting bracket includes a passageway that defines a second longitudinal axis that is offset in a lateral direction from, and extends parallel to, the first longitudinal axis. The orthopaedic surgical instrument system also comprises an elongated shaft extending through the housing from a shank positioned adjacent to the superior end of the housing to a shaft end positioned adjacent to the mounting bracket, and a reamer including a head coupled to the shaft end and a plurality of cutting teeth defined on a bone-facing surface of the head. A medial-lateral distance is defined between the first longitudinal axis and the second longitudinal axis, and the reamer head has a circular outer circumference and a radius that is less than the medial-lateral distance.

In some embodiments, the elongated shaft may be configured to slide axially within the housing along the first longitudinal axis. In some embodiments, the mounting bracket may include a medial pocket, and the reamer head may extend into the medial pocket.

In some embodiments, a tab may extend inferiorly from a planar surface of the mounting bracket. In some embodiments, the housing and the mounting bracket are formed as a single, monolithic component.

According to another aspect, a method of performing a surgical procedure is disclosed. The method comprises impacting a femoral broach into a proximal opening of a femoral canal of a patient's femur, aligning a mounting bracket of a reamer assembly with a proximal post of the femoral broach, advancing the mounting bracket over the proximal post to position cutting teeth of the reamer assembly in contact with a medial calcar of the patient's femur, and reaming the medial calcar with the cutting teeth of the reamer assembly. During the performance of the method, all of the cutting teeth of the reamer assembly are positioned medial of the proximal post of the femoral broach.

In some embodiments, advancing the mounting bracket over the proximal post may include moving the cutting teeth relative to the mounting bracket to position the cutting teeth in contact with the medial calcar of the patient's femur. In some embodiments, advancing the mounting bracket over the proximal post may include offsetting a reamer head of the reamer assembly in a medial direction from a lateral edge of the femoral broach to position the reamer head above a medial edge of the femoral broach.

According to another aspect, a method of performing a surgical procedure is disclosed. The method comprises inserting a first orthopaedic surgical instrument into a proximal opening of a femoral canal of a patient's femur, aligning a mounting bracket of a reamer assembly with a post of the first orthopaedic surgical instrument, advancing the mounting bracket over the post to position cutting teeth of the reamer assembly in contact with a medial calcar of the patient's femur, and reaming the medial calcar with the cutting teeth of the reamer assembly. During the performance of the method, all of the cutting teeth of the reamer assembly are positioned medial of the post of the first orthopaedic surgical instrument.

### BRIEF DESCRIPTION OF THE DRAWINGS

FIG. 1 is a perspective view of an orthopaedic surgical instrument configured to resect a medial calcar of a patient's femur;

FIG. 2 is another perspective view of the orthopaedic surgical instrument of FIG. 1;

FIG. 3 is a side elevation view of another orthopaedic surgical instrument for use with the orthopaedic surgical instrument of FIG. 1;

FIG. 4 is a perspective view of one end of the orthopaedic surgical instrument of FIG. 3;

FIG. 5 is a side elevation view of a femoral stem component;

FIG. 6 is an exploded side elevation view of the orthopaedic surgical instruments of FIGS. 1-4 positioned relative to a patient's femur;

FIGS. 7-8 are perspective views of the orthopaedic surgical instruments of FIGS. 1-4 coupled together;

FIG. 9 is a side elevation view similar to FIG. 6 showing the orthopaedic surgical instruments of FIGS. 1-4 during a resection of the medial calcar; and

FIG. 10 is a perspective view showing the relative dimensions of the reamer head of the orthopaedic surgical instrument of FIG. 1 and the collar of the femoral stem component of FIG. 5.

### DETAILED DESCRIPTION

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, etcetera, may be used throughout the specification in reference to the orthopaedic implants or prostheses and surgical instruments described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise.

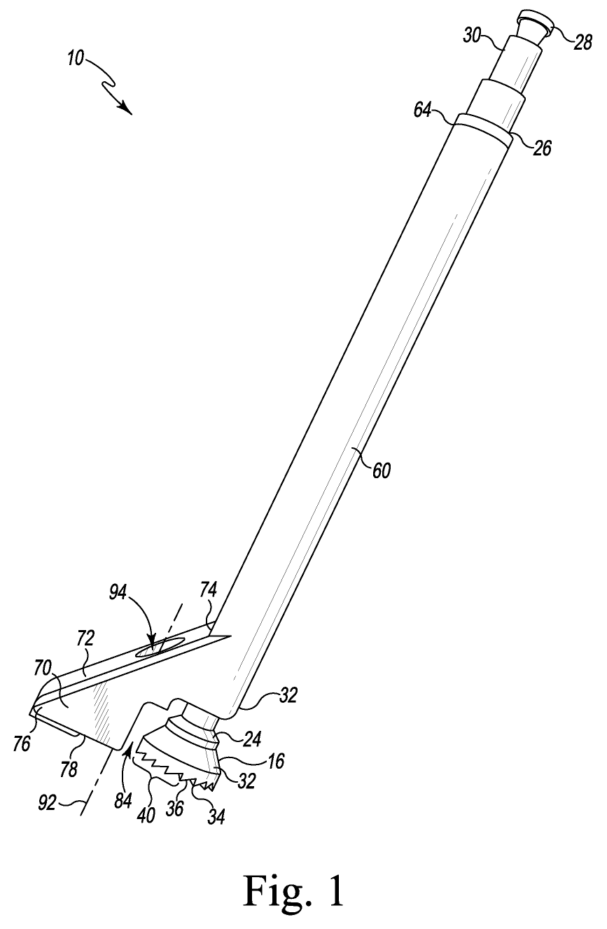

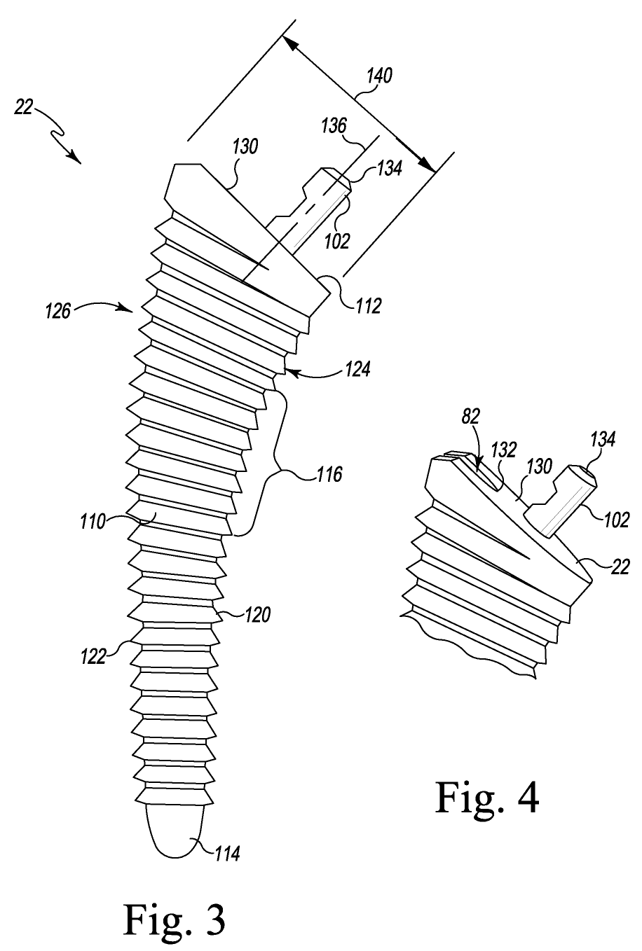

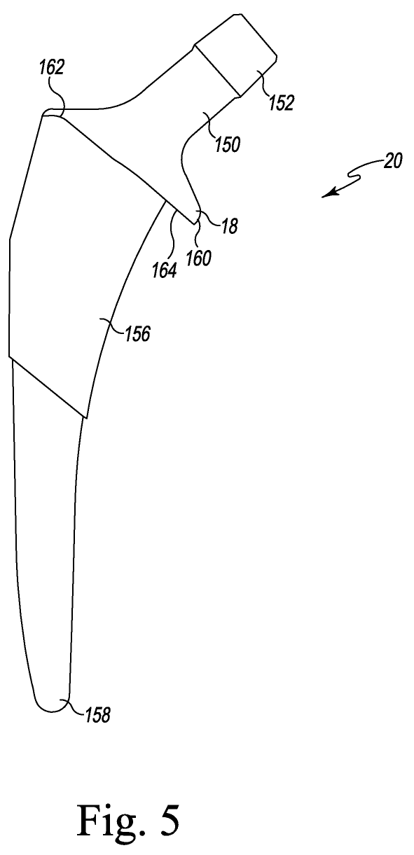

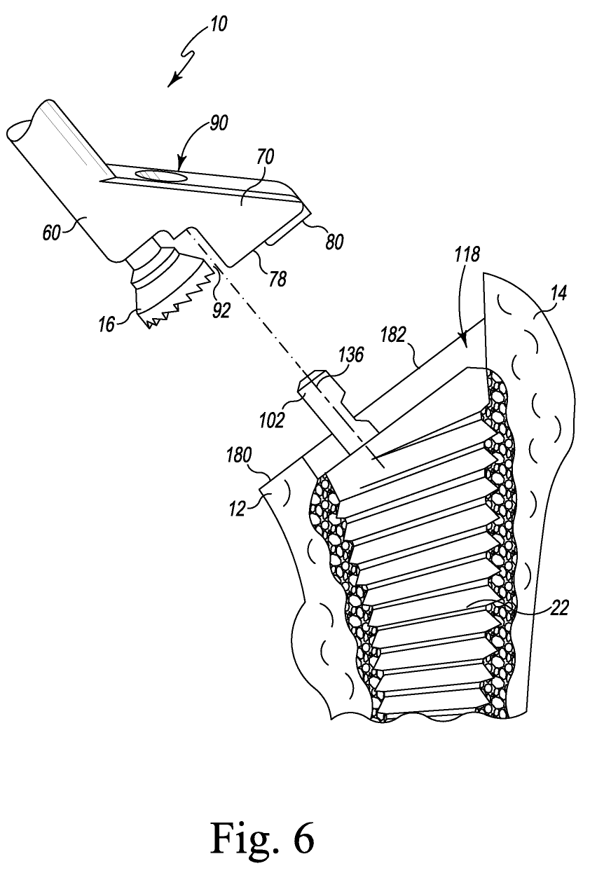

Referring now to FIG. 1, an orthopaedic surgical instrument 10 configured to resect a medial calcar 12 (see FIG. 6) of a patient's femur 14 is shown. In the illustrative embodiment, the instrument 10 is a reamer assembly that includes a reamer head 16 sized to limit the resection to the region in which a collar 18 (see FIG. 5) of a femoral stem component 20 will be present when the femoral stem component 20 is implanted into the surgically-prepared femur. As described in greater detail below, the reamer assembly 10 is configured to be coupled to another orthopaedic surgical instrument—a femoral broach 22 (see FIG. 3)—which acts as a mount and resection guide. In that way, the illustrative embodiment also accounts for the degree of version, anterior-posterior angulation, and varus/valgus of the implanted femoral stem component 20.

The reamer head 16 of the orthopaedic surgical instrument 10 is coupled to a lower end 24 of an elongated shaft 26. The reamer head 16 and the shaft 26 are formed from metallic materials such as, for example, stainless steel, which may be autoclaved and sterilized between surgical procedures. As shown in FIG. 1, the elongated shaft 26 extends from the lower end 24 to an upper end 28. A tool shank 30 configured to be coupled to a rotary surgical tool such as, for example, a surgical drill, is defined at the upper end 28 of the shaft 26. In the illustrative embodiment, the shaft 26 and the reamer head 16 are formed as separate components that are later assembled. In other embodiments, the shaft 26 and the reamer head 16 may be formed as a single monolithic component. It should also be appreciated that in other embodiments other materials may be used. For example, portions of the reamer head and/or shaft may be formed from a polymeric material such as polyethylene.

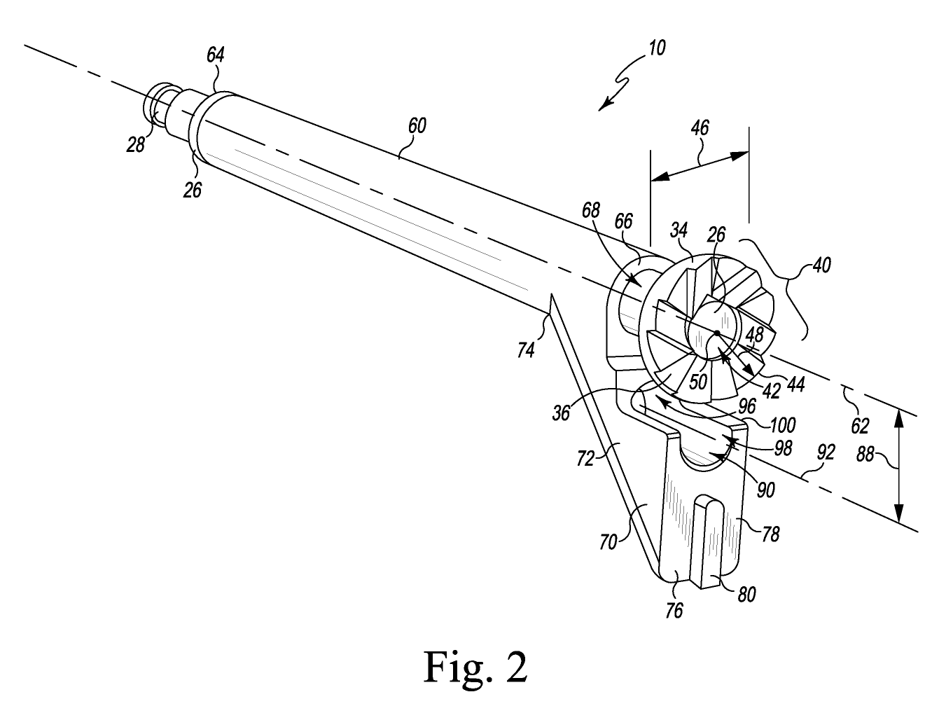

As shown in FIGS. 1-2, the reamer head 16 includes a frustoconical body 32 that extends from the lower end 24. The reamer head 16 also has an outer rim wall 34 that extends from the body 32 to a bottom surface 36 of the reamer head 16. In the illustrative embodiment, a plurality of cutting teeth 40 are defined in the bottom surface 36 to resect portions of the patient's bone. As shown in FIG. 2, the cutting teeth 40 surround a cavity 42 defined in the center of the bottom surface 36. In the illustrative embodiment, the elongated shaft 26 extends into the cavity 42 to secure the reamer head 16 to the shaft.

The bottom surface 36 of the reamer head 16 and the rim wall 34 meet at a circular outer edge 44 in the illustrative embodiment that defines the outer circumference of the reamer head 16. As shown in FIG. 2, the bottom surface 36 has an outer diameter 46, and a radius 48 is defined between an origin 50 positioned at the center of the bottom surface 36 and the outer edge 44. The outer diameter 46 is 18 millimeters in the illustrative embodiment. The corresponding radius 48 is 9 millimeters in the illustrative embodiment. It should be appreciated that the reamer assembly 10 may be included in a kit with a number of reamer assemblies having heads of different sizes, which correspond to a range of implant sizes.

The elongated shaft 26 extends through a housing 60 of the orthopaedic surgical instrument 10. In the illustrative embodiment, the elongated shaft 26 is coupled to the housing 60 via, for example, a tongue and slot arrangement (not shown), which permits the elongated shaft 26 to slide axially relative to the housing 60 to change the position of the reamer head 16. The elongated shaft 26 (and hence the reamer head 16) is also configured to rotate about a rotational axis 62 relative to the housing 60. As shown in FIG. 2, the axis 62 is a longitudinal axis extending along the elongated shaft 26 through the center of the reamer head 16 (including the origin 50).

The housing 60 extends from an upper end 64 positioned adjacent to the upper end 28 of the elongated shaft 26 to a lower end 66 shown positioned adjacent to the lower end 24 in FIG. 2. The housing 60 has a cylindrical passageway 68 sized to receive the elongated shaft 26, which extends through openings defined in its ends 64, 66.

The reamer assembly 10 also includes a mounting bracket 70 that extends laterally from the lower end 66 of the housing 60. In the illustrative embodiment, the bracket 70 and the housing 60 are formed as a single monolithic component from a metallic material such as, for example, stainless steel, which may be autoclaved and sterilized between surgical procedures. It should be appreciated that in other embodiments other materials may be used and the bracket and the housing may be formed separately and later assembled.

The mounting bracket 70 has a body 72 that extends from a base 74 attached to the lower end 66 of the housing 60 to a tip 76. The tip 76 has a planar bottom surface 78, and the mounting bracket 70 includes an alignment tab 80 that extends outwardly from the surface 78. In the illustrative embodiment, the alignment tab 80 is shaped to engage a corresponding alignment groove 82 (see FIG. 4) defined in the femoral broach 22 to position the reamer assembly 10 in a single orientation relative to the broach. The mounting bracket 70 also includes a pocket 84, which is defined in the body 72 below the lower end 66 of the housing 60. As shown in FIG. 2, the reamer head 16 extends into the pocket 84.

The mounting bracket 70 also includes a passageway 90 that extends through the body 72 along a longitudinal axis 92. As shown in FIG. 2, the longitudinal axis 92 (and hence the passageway 90) is offset from the rotational axis 62 and extends parallel to it. The longitudinal axis 92 and the rotational axis 62 are separated by a medial-lateral distance 88 that is greater than the radius 48 of the reamer head 16. In that way, the portion of the edge 44 of the reamer head that is positioned in the pocket 84 is positioned between the axes 62, 92. Additionally, the reamer head 16 is offset medially from the passageway 90.

The passageway 90 of the mounting bracket 70 includes an upper section 94 that is defined by a cylindrical wall 96. The upper section 94 opens into the pocket 84 and is connected to a lower section of the passageway 90. In the illustrative embodiment, the lower section is a groove 98 defined in a sidewall 100 of the body 72 that faces the pocket 84. As shown in FIG. 2, the groove 98 (and hence the passageway 90) extends through the bottom surface 78 of the bracket 70. As described in greater detail below, the passageway 90 is sized to receive a post 102 of the femoral broach 22 to couple the instrument 10 to the broach 22.

Referring now to FIG. 3, the femoral broach 22 includes an elongated body 110 that extends from a proximal end 112 to a distal tip 114. A plurality of cutting teeth 116 are defined along the length of the body 110, and each tooth 116 is shaped and sized to surgically-prepare a femoral canal 118 (see FIG. 6) of the patient's femur 14 to receive the femoral stem component 20. When the femoral broach 22 is viewed in a coronal plane as shown in FIG. 3, the body 110 has a medial edge 120 that extends from the proximal end 112 to the distal tip 114 and a lateral edge 122 that is positioned opposite the medial edge 120. The medial edge 120 has a concave section 124 that is positioned adjacent to the proximal end 112 of the body 110, and the lateral edge 122 has a corresponding convex section 126 that is positioned opposite the concave section 124. The curvatures of the sections 124, 126 correspond to the curvatures of the corresponding regions of the femoral stem component 20.

The femoral broach 22 includes a planar proximal surface 130 at the proximal end 112 of the elongated body 110. As described above, the broach 22 includes an alignment groove 82, which defined in the planar proximal surface 130. As shown in FIG. 4, the groove 82 extends inwardly from the lateral edge 122 of the broach 22 to a closed end 132. The broach 22 also includes a proximal post 102 that extends outwardly from the surface 130 to a tip 134. In the illustrative embodiment, the post 102 extends along a longitudinal axis 136, which extends orthogonal to the planar surface 130. In the illustrative embodiment, the planar surface 130 has a medial-lateral width 140 that is defined between the medial edge 120 and the lateral edge of the broach 22.

In the illustrative embodiment, the femoral broach 22 is formed as a single monolithic component from a metallic material such as stainless steel. It should also be appreciated that in other embodiments other materials may be used. For example, portions of the post and/or elongated body may be formed from a polymeric material such as polyethylene, while the edges of the cutting teeth are formed from a metallic material.

Referring now to FIG. 5, a femoral stem component 20 of a hip prosthesis is shown. The femoral stem component 20 is configured to be inserted into a femoral medullary canal 118 of a patient's femur 14 that has been prepared using the femoral broach 22 and the reamer assembly 10. The stem component 20 includes a neck 150 configured to be coupled to a head component (not shown) to engage a patient's natural acetabulum (not shown) or a prosthetic acetabular cup implanted into the patient's pelvic bone. In the illustrative embodiment, the neck 150 includes a tapered trunnion 152 configured to engage with a tapered aperture defined in the head component to secure the components together. It should be appreciated that in other embodiments the neck and the head component may be configured to be threaded, press fit, or secured together by other fastening means.

The stem component 20 also includes a collar 18 and an elongated body 156 that extends distally from the collar to a distal tip 158. As shown in FIG. 1, the neck 150 extends medially and proximally from the collar 18. In the illustrative embodiment, the neck 150, the collar 18, and the elongated body 156 are formed as a monolithic structure (e.g., a single molded or cast part). It should be appreciated that in other embodiments these components may be formed as separate components secured to one another by a mechanical fastener (e.g., screw, bolt, taper fit, etc.), adhesive, or other suitable fastener. In some embodiments, the stem component 20 may also include a porous outer coating that extends over some or all of the elongated body 156.

As shown in FIG. 5, the collar 18 extends outwardly to a medial tip 160 that is spaced apart from the proximal end 162 of the elongated body 156 and the neck 150. The collar 18 also includes a planar distal surface 164 that extends medially from. the proximal end 162 of the body 156 to the tip 160. As described in greater detail below, the distal surface 164 is configured to engage the medial calcar 12 of the patient's femur 14 after it has been surgically-prepared by the reamer assembly 10.

Referring now to FIGS. 6-9, the reamer assembly 10 and the femoral broach 22 are shown during a procedure to prepare the patient's femur 14 to receive the femoral stem component 20. During the surgical procedure, a surgeon or other member of the surgical team may resect the patient's femur 14 to remove the natural femoral head and create a substantially planar proximal surface 180 on the patient's femur 14, which is shown in FIG. 6. The surgeon may use an osteotome to create an opening 182 into the femoral canal 118.

The surgeon may then insert the femoral broach 22 through the opening 182 into the canal 118 and advance the broach 22 distally along the canal 118 to size and shape the canal 118 to receive the femoral stem component 20. If the surgeon determines that the femoral broach is not axially and rotationally stable when seated in the canal, the surgeon may withdraw the first broach 22 from canal 118, select another broach 22 that is larger in size, and insert that broach 22 into the canal 118. The surgeon may continue to increase broach sizes until the selected broach 22 attains axial and rotational stability and is seated at the level shown in, for example, FIG. 6, which recreates proper leg length for the patient. The surgeon may then perform a trial reduction with the broach 22 seated in the canal 118, assessing, for example, component position, joint stability, range of motion, and leg length.

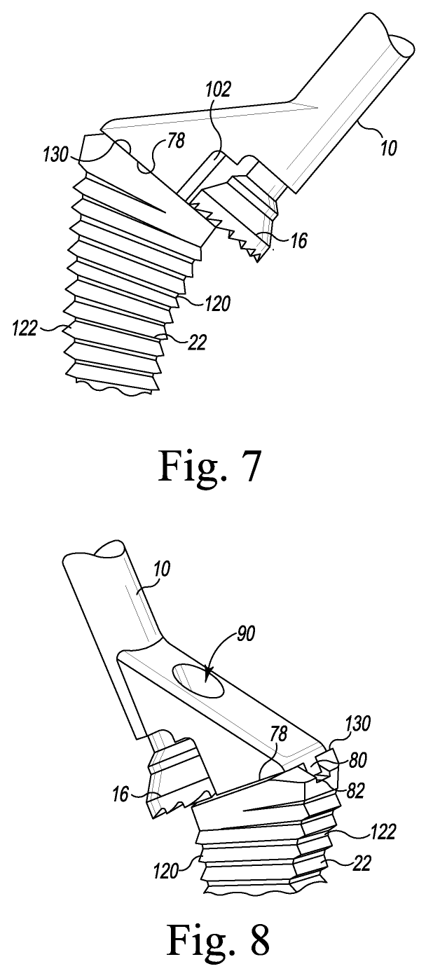

With the broach 22 seated in the canal 118, the surgeon may utilize the reamer assembly 10 to resect the medial calcar 12 of the patient's femur 14. To do so, the surgeon may align the passageway 90 of the mounting bracket 70 of the reamer assembly with the proximal post 102 of the broach 22. The surgeon may also rotate the reamer assembly 10 to align the alignment tab 80 with the groove 82 of the broach 22. With the reamer assembly 10 properly oriented, the surgeon may advance the reamer assembly 10 distally to position the mounting bracket 70 over the post 102 and move the alignment tab 80 into the groove 82. As shown in FIGS. 7-8, the bottom surface 78 of the mounting bracket 70 contacts the proximal surface 130 of the broach 22 when the reamer assembly 10 is properly coupled to the broach.

As described above, the reamer head 16 sized and positioned to limit the resection to the region in which the collar 18 of the femoral stem component 20 will be present when the femoral stem component 20 is implanted into the surgically-prepared femur. In the illustrative embodiment, the reamer head 16 has a diameter 46 that is defined by its outer circumference, and this diameter 46 is less than the medial-lateral width 140 of the proximal surface 130 of the broach 22. Additionally, the reamer head 16 is offset medially from the lateral edge 122 of the broach 22 and is positioned above the medial edge 120 when the reamer assembly is coupled to the broach. As shown in FIGS. 7-8, the reamer head 16 is also positioned medial of the post 102 of the broach 22 such that the resection is limited to a region of the proximal surface 180 of the patient's bone that is medial of the post 102, i.e., region of the medial calcar 12 of the patient's femur.

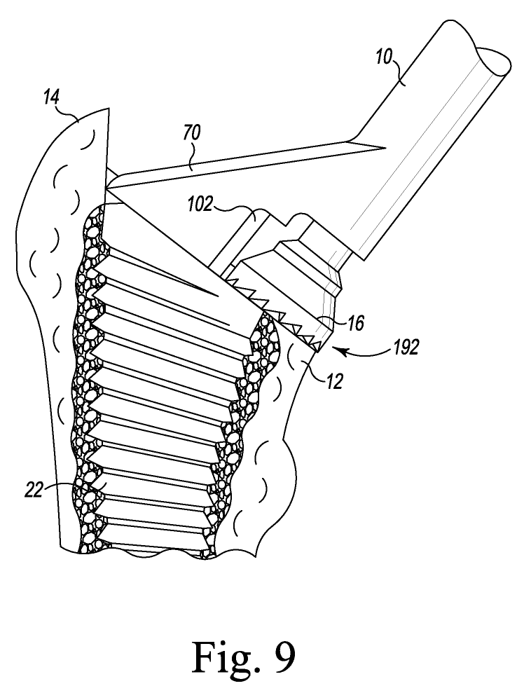

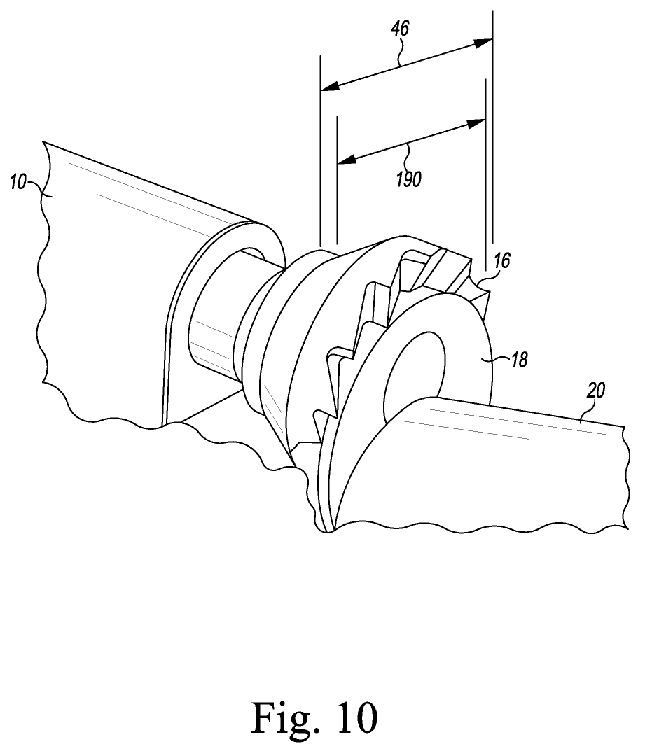

The surgeon may connect a surgical drill to the tool shank 30 of the reamer assembly 10. The surgeon may then operate the drill to rotate the elongated shaft 26 and the reamer head 16 about the axis 62 to resect the medial calcar 12. As described above, the elongated shaft 26 is configured to slide axially in an inferior-superior direction relative to the housing 60 such that the reamer head 16 may be advanced downward as the patient's bone is resected. In embodiments in which the shaft 26 does not move relative to the mounting bracket, reaming may begin with the mounting bracket 70 spaced apart from the proximal surface 130 of the broach 22 and the entire reamer assembly 10 may advance downward as bone material is removed. As shown in FIG. 9, the resection is complete when the cutting teeth 40 of the reamer head 16 engage the proximal surface 130 of the broach 22.

Referring now to FIG. 10, the cutting diameter 46 of the reamer head 16 is slightly greater than the anterior-poster width 190 of the collar 18 of the stem component 20. In that way, the reamer head 16 is configured to define a groove or pocket 192 in the medial calcar 12 sized to receive the collar 18. It should be appreciated that the groove and collar create a rotational lock between the stem component 20 and the patient's femur 14, which inhibits relative rotation between the component 20 and the patient's femur 14.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected. For example, it should be appreciated that the reamer assembly may be configured for use with other orthopaedic surgical instruments. In one such embodiment, the reamer assembly may be configured to be coupled to a stem reamer inserted in the patient's femur. Such stem reamers may be used to resect distal portions of the femoral canal, and the reamer assembly may be configured to be coupled to the reamer shaft about the reamer's cutting teeth.

There are a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

1. An orthopaedic surgical instrument system, comprising:
a first orthopaedic surgical instrument including an elongated body sized to be positioned in a femoral canal of a patient's femur and a plurality of cutting teeth defined in the elongated body, and
a second orthopaedic surgical instrument including a reamer head configured to resect a portion of a proximal end of the patient's femur and a mounting bracket configured to secure the reamer head to a proximal end of the first orthopaedic surgical instrument,
wherein the elongated body of the first orthopaedic surgical instrument includes a medial edge and a lateral edge positioned opposite the medial edge, and
wherein when the mounting bracket is coupled to the proximal end of the first orthopaedic surgical instrument, an outer circumference of the reamer head is offset medially from the lateral edge of the elongated body such that the reamer head is positioned to resect a medial calcar of the patient's femur.

2. The orthopaedic surgical instrument system of claim 1, wherein the reamer head is positioned above only the medial edge of the elongated body when the mounting bracket is coupled to the proximal end of the first orthopaedic surgical instrument.

3. The orthopaedic surgical instrument system of claim 1, wherein the reamer head is coupled to the mounting bracket such that the reamer head is permitted to move in an inferior-superior direction relative to the elongated body of the first orthopaedic surgical instrument when the mounting bracket is coupled to the proximal end of the first orthopaedic surgical instrument.

4. The orthopaedic surgical instrument system of claim 1, wherein the elongated body of the first orthopaedic surgical instrument includes a proximal planar surface that has a medial-lateral width, and the outer circumference of the reamer head has a diameter less than the medial-lateral width of the proximal planar surface.

5. The orthopaedic surgical instrument system of claim 4, wherein the first orthopaedic surgical instrument includes a post extending outwardly from the proximal planar surface, and the mounting bracket is configured to be coupled to the post.

6. The orthopaedic surgical instrument system of claim 5, wherein the mounting bracket of the second orthopaedic surgical instrument includes a cylindrical passageway that is sized to receive the post.

7. The orthopaedic surgical instrument system of claim 6, wherein the cylindrical passageway extends along a longitudinal axis, and the reamer head is configured to rotate about a rotational axis extending parallel to the longitudinal axis.

8. The orthopaedic surgical instrument system of claim 7, wherein the second orthopaedic surgical instrument further includes an elongated shaft extending along the rotational axis from the reamer head to a shank shaped to be coupled to a rotary surgical instrument.

9. The orthopaedic surgical instrument system of claim 8, wherein the second orthopaedic surgical instrument includes a housing connected to the mounting bracket, and the elongated shaft of the second orthopaedic surgical instrument extends through the housing to the shank positioned outside of the housing.

10. The orthopaedic surgical instrument system of claim 4, wherein:
the first orthopaedic surgical instrument includes a slot defined in the proximal planar surface, and
the mounting bracket of the second orthopaedic surgical instrument includes a tab sized to be received in the slot to prevent rotation of the mounting bracket relative to the mounting bracket relative to the first orthopaedic surgical instrument.

11. The orthopaedic surgical instrument system of claim 1, wherein the first orthopaedic surgical instrument is a femoral broach.

12. The orthopaedic surgical instrument system of claim 1, wherein the medial edge of the elongated body of the first orthopaedic surgical instrument includes a concave section and the lateral edge of the elongated body includes a convex section positioned opposite the concave section.

13. The orthopaedic surgical instrument system of claim 12, wherein the elongated body extends to a distal tip.

14. An orthopaedic surgical instrument system, comprising:
a housing extending along a first longitudinal axis from a superior end to an inferior end,
a mounting bracket extending from the inferior end of the housing, the mounting bracket including a passageway that defines a second longitudinal axis that is offset in a lateral direction from, and extends parallel to, the first longitudinal axis,
an elongated shaft extending through the housing from a shank positioned adjacent to the superior end of the housing to a shaft end positioned adjacent to the mounting bracket, and
a reamer including a head coupled to the shaft end and a plurality of cutting teeth defined on a bone-facing surface of the head,
wherein a medial-lateral distance is defined between the first longitudinal axis and the second longitudinal axis, and the reamer head has a circular outer circumference and a radius that is less than the medial-lateral distance.

15. The orthopaedic surgical instrument system of claim 14, wherein the elongated shaft is configured to slide axially within the housing along the first longitudinal axis.

16. The orthopaedic surgical instrument system of claim 14, wherein the mounting bracket includes a medial pocket, and the reamer head extends into the medial pocket.

17. The orthopaedic surgical instrument system of claim 14, wherein a tab extends inferiorly from a planar surface of the mounting bracket.

18. A method of performing a surgical procedure, the method comprising:
impacting a femoral broach into a proximal opening of a femoral canal of a patient's femur,
aligning a mounting bracket of a reamer assembly with a proximal post of the femoral broach,
advancing the mounting bracket over the proximal post to position cutting teeth of the reamer assembly in contact with a medial calcar of the patient's femur, and
reaming the medial calcar with the cutting teeth of the reamer assembly,
wherein all of the cutting teeth of the reamer assembly are positioned medial of the proximal post.

19. The method of claim 18, wherein advancing the mounting bracket over the proximal post includes moving the cutting teeth relative to the mounting bracket to position the cutting teeth in contact with the medial calcar of the patient's femur.

20. The method of claim 18, wherein advancing the mounting bracket over the proximal post includes offsetting a reamer head of the reamer assembly in a medial direction from a lateral edge of the femoral broach to position the reamer head above a medial edge of the femoral broach.
